(19) United States
(12) Patent Application Publication
(10) Pub. No.: US 2026/0240682 A1
(43) Pub. Date: Aug. 20, 2026

(54) A SUPRACHOROIDAL SPACER IMPLANT TO TREAT GLAUCOMA

(71) Applicant: The Board of Trustees of the Leland Stanford Junior University, Stanford, CA (US)

(72) Inventors: Jeffrey Louis Goldberg, Menlo Park, CA (US); David Myung, Saratoga, CA (US); Bryce Chiang, Redwood City, CA (US)

(21) Appl. No.: 19/489,498

(22) PCT Filed: May 31, 2024
(86) PCT No.: PCT/US2024/032027

§ 371 Date: Dec. 2, 2025

Related U.S. Application Data
(60) Provisional application No. 63/471,438, filed on Jun. 6, 2023.

(51) Int. Cl. A61F 9/007; A61L 27/16; A61L 27/52; A61L 27/54

(52) U.S. Cl. CPC A61F 9/00781; A61L 27/16; A61L 27/52; A61L 27/54; A61L 2430/16

(57) ABSTRACT

Composition and methods are provided for the treatment of ocular hypertension or glaucoma, wherein intraocular pressure (IOP) is lowered by insertion of an implant suprachoroidal spacer into the suprachoroidal space

### CROSS REFERENCE TO OTHER APPLICATIONS

This application claims the benefit of U.S. Provisional Application No. 63/471,438 filed Jun. 6, 2023, the contents of which are hereby incorporated by reference in its entirety.

### GOVERNMENT SUPPORT RESEARCH

This invention was made with Government support under contract EY026877 awarded by the National Institutes of Health. The Government has certain rights in the invention.

### BACKGROUND

Glaucomatous optic neuropathy (aka, glaucoma) is a leading cause of blindness in the United States and worldwide. The optic nerve is formed by 1.5 million retinal ganglion cell axons as they coalesce to leave the eye and head towards the brain, carrying information that is eventually processed as vision. Glaucoma is characterized by progressive cupping of the optic nerve head, which is perceived by glaucoma patients as slow, painless, and progressive loss of peripheral vision eventually involving central vision. Intraocular pressure (IOP) is determined by the balance of aqueous humor production and elimination, with a population average of 14.5 mmHg as “normal” and >21 mmHg as “abnormal” (2 standard deviations above normal).

Elevated IOP has been identified as the only modifiable risk factor in glaucoma, and IOP reduction of 20-30% has been shown to slow or halt glaucoma progression. However, not all patients with abnormally elevated IOP will exhibit progressive changes, and thus do not have glaucoma. On the other hand, there exists a subclass of glaucoma with normal IOP, termed normal tension glaucoma or NTG. IOP reduction of 20-30% in NTG patients (from “normal” IOP to low-normal IOP) has also been shown to slow disease progression. Thus, there seems to be a personalized IOP threshold for each individual glaucoma patient where glaucomatous damage ceases.

IOP reduction can be achieved through topical eye drops, with laser procedures, and glaucoma surgeries. Topical eyedrops with various mechanisms of actions (such as to lower aqueous production and/or increase outflow) are effective and generally well tolerated, but medication adherence with eyedrops is low, likely related to increasingly complex medication regimens, dexterity, and/or memory problems. Laser procedures can be effective but the magnitude and duration of IOP reduction is unpredictable. Glaucoma surgeries can either augment or totally replace aqueous outflow. As with all surgeries, there are risks associated with surgery including bleeding, infection, IOP too high or too low, further vision loss, diplopia, etc. Some of these complications can be devastating and result in total loss of vision and/or the eye. In addition, the timing of glaucoma surgery can be challenging. There exists a need for a glaucoma procedure that is safe, efficacious, and can be performed in the outpatient clinic setting.

Suprachoroidal expansion has been shown to reduce IOP in rabbits and rhesus macaques. The suprachoroidal space is a potential space between the sclera and the choroid that has become an attractive option for targeted ocular drug delivery. Expansion of this space is thought to enhance outflow and/or reduce aqueous humor production. The degree of suprachoroidal expansion was associated with IOP reduction. Surprisingly, expansion with silicone oil did not lower IOP suggesting that surface tension of material injected is important, as this would impact the ability of aqueous humor to freely traverse through the expanded space.

### SUMMARY

Composition and methods are provided for the treatment of ocular hypertension, and/or glaucoma, wherein intraocular pressure (IOP) is lowered by insertion of an implant suprachoroidal spacer into the suprachoroidal space with a specially designed injector. In some embodiments the implant is a monolithic implant. Spacer implants are inserted parallel or perpendicular to the limbus. The suprachoroidal space is a potential space between the sclera and the choroid held together by fibrils, and thus a nominal force is required to part the tissue. Thus implant is rigid enough so as to easily advance within the suprachoroidal space but not so rigid to inadvertently penetrate through the choroid and retina.

Suprachoroidal space expansion has been shown to lower IOP, but prior art injectable formulations have dissolved within months, reducing their desirability. The monolithic suprachoroidal spacer implant of the disclosure is designed to be long lasting, so as to be effective for a period of at least about 1 year, 2 years, 3 years or more. The injector has been designed to enable the implant to be safely delivered with an outpatient office-based procedure.

In some embodiments the implant has final dimensions of from about 0.5 mm to about 1.5 mm diameter when hydrated, e.g. from about 0.5, 0.6, 0.7, 0.8, 0.9, 1 mm to about 1.5, 1.4, 1.3, 1.2, 1.1 mm. The length may be from about 5 mm to about 15 mm in length, e.g. from about 5, 6, 7, 8, 9, 10 mm, up to about 15, 14, 13, 12, 11 mm.

In some embodiments the material of an implant is an inert, biocompatible, water permeable material of appropriate rigidity. Suitable materials include, for example, hydrogels such as PEG methacrylate, hyaluronic acid, etc; polyvinyl alcohol (PVA); cellulose; collagen; polypropylene; polyurethane; nylon; PLGA, PLA, polycaprolactone, etc. In some embodiments the implant is a PVA hydrogel. In some embodiments the implant is an open cell polypropylene foam. In some embodiments the spacer implant is dip coated in sucrose, hyaluronic acid, polyvinyl alcohol, carboxmymethylcellulose, etc. to strengthen and/or facilitate insertion.

In some embodiments the implant is a metallic implant, e.g. formed of nitinol, steel, aluminum, and other biocompatible alloys. In some embodiments the implant is a plastic. In some such embodiments the implant may be coated, e.g. with an inert, biocompatible, water permeable material. In some embodiments the coating is a hydrogel, including PEG, PEG methacrylate; polyvinyl alcohol (PVA); cellulose; collagen; polypropylene; polyurethane; PEEK; nylon, etc.

In some embodiments an implant or an implant coating comprises a drug, e.g. for sustained release. Drugs of interest for this purpose may include, without limitation, one or more of: β-blockers such as timolol, and betaxolol; alpha-agonists, such as brimonidine; carbonic anhydrase inhibitors such as dorzolamide, and brinzolamide; prostaglandin analogues such as latanoprost, bimatoprost, travoprost, tafluprost, latanoprostene bunod, etc.; rho kinase inhibitors, such as netarsudil; antibiotics such as moxifloxacin, cefalexin, cefazolin, etc.; enzymes such as protease, matrix metalloproteinase, collagenase, etc.; and the like.

The injector is designed to safely and reliably access the suprachoroidal space using a microneedle with length matched to the thickness of the sclera, for example a 27-gauge needle (OD 0.406 mm), which does not require suturing of the sclerotomy to prevent leakage and/or infection. The microneedle may be micromachined to a length appropriate for the insertion, e.g. from about 0.5 to about 1.5 mm in length. The baseplate may be angled, e.g. from about 45° to about 90°, usually about 65°. No cyclodialysis cleft is needed for this device to have its effect.

In some embodiments an injector device is provided, optionally including a pre-loaded spacer implant. In other embodiments, a kit comprising each of the injector device and spacer implants are provided, which kit may further comprise reagents and instructions for use.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1 Photo of custom designed injector (pictured with 25° bevel angle and #000 washer.

FIG. 2 Representative PEG hydrogel block (in this case 30% 10 kDa 4-arm PEG-methacrylate) before (top) and after (bottom) compression testing.

FIG. 3 Graphs of stiffness (Young's modulus at 5% strain) for PEG hydrogels. Unless otherwise specified, 40% (w/v) 4-arm PEG-methacrylate 10 kDa was used. (A) Varying molecular weight. (B) Varying functional group. (C) Varying molecular weight.

FIG. 4 The effect of needle length and insertion angle in positioning the implant within the suprachoroidal was found. (A) Schematic of experiment demonstrating insertion angle and needle length. The tip was found to be outside the eye (yellow V), within the suprachoroidal space (SCS, green O), or through the retina (red X). (B) Each square represents one replicate.

FIG. 5 (A) Probability of staying within the suprachoroidal space (SCS). (B) Bend force for different prolene sutures when held at 5 mm.

FIG. 6. (A) PEG hydrogel being molded in silicone tubing. (B) PEG hydrogel implant. (C) Bending strength of implant, demonstrating effect of different parameters.

FIG. 7. (A) Microneedle injection to deliver viscoelastic. (B) implant inserter. (C) IOP difference (treated−untreated) over time for 3 rabbits.

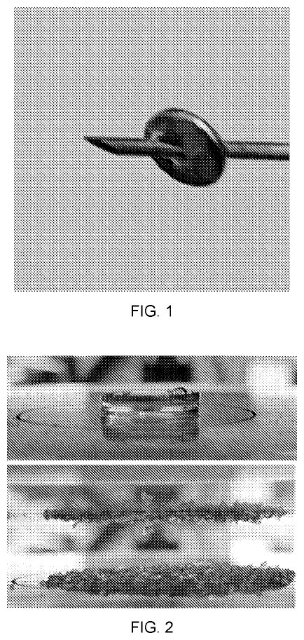

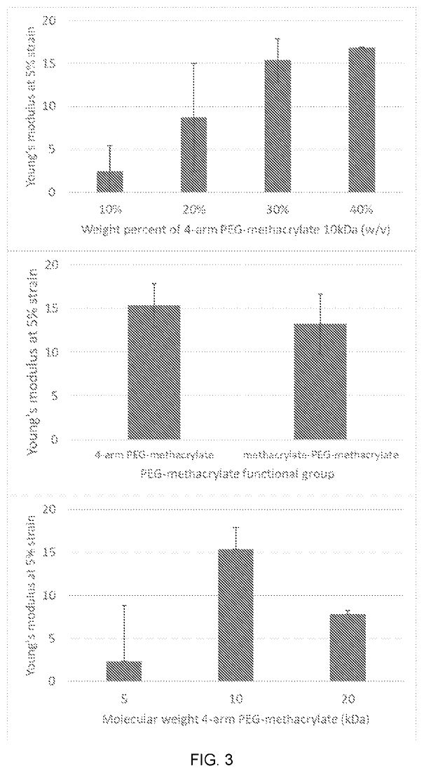

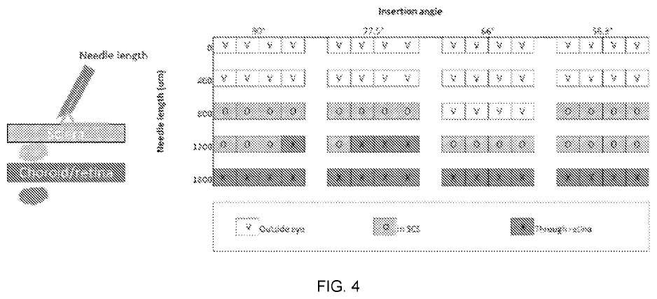

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As used herein, compounds which are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

Compounds can also be made by methods known to one of ordinary skill in the art. As used herein, “methods known to one of ordinary skill in the art” may be identified though various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

“Inhibiting” the onset of a disorder shall mean either lessening the likelihood of the disorder's onset, or preventing the onset of the disorder entirely. In the preferred embodiment, inhibiting the onset of a disorder means preventing its onset entirely.

“Treating” a disorder shall mean slowing, stopping or reversing the disorders progression. In the preferred embodiment, treating a disorder means reversing the disorder's progression, ideally to the point of eliminating the disorder itself. As used herein, ameliorating a disorder and treating a disorder are equivalent. The effect may be prophylactic in terms of completely or partially preventing a disease or symptom thereof and/or may be therapeutic in terms of effecting a partial or complete cure for a disease and/or symptoms of the disease.

The terms “subject,” “individual,” and “patient” are used interchangeably herein to refer to a mammal being assessed for treatment and/or being treated. In an embodiment, the mammal is a human. The terms “subject,” “individual,” and “patient” thus encompass individuals having fibrosis, including without limitation, tumor fibrosis, cardiac fibrosis, liver fibrosis, kidney fibrosis, lung fibrosis, dermal scarring and keloids, Alzheimers disease, etc. Subjects may be human, but also include other mammals, particularly those mammals useful as laboratory models for human disease, e.g. mouse, rat, etc.

“Suitable conditions” shall have a meaning dependent on the context in which this term is used. In one embodiment, the term “suitable conditions” as used herein means physiological conditions.

“In combination with”, “combination therapy” and “combination products” refer, in certain embodiments, to the concurrent administration to a patient of a first and second therapeutic. When administered in combination, each component can be administered at the same time or sequentially in any order at different points in time. Thus, each component can be administered separately but sufficiently closely in time so as to provide the desired therapeutic effect.

“Concomitant administration” of agents means administration of the agents at such time that both will have a therapeutic effect. Such concomitant administration may involve concurrent (i.e. at the same time), prior, or subsequent administration. A person of ordinary skill in the art would have no difficulty determining the appropriate timing, sequence and dosages of administration for particular agents of the present invention.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

The phrase “determining the treatment efficacy” and variants thereof can include any methods for determining that a treatment is providing a benefit to a subject. The term “treatment efficacy” and variants thereof are generally indicated by alleviation of one or more signs or symptoms associated with the disease and can be readily determined by one skilled in the art. “Treatment efficacy” may also refer to the prevention or amelioration of signs and symptoms of toxicities typicaly associated with standard or non-standard treatments of a disease. Determination of treatment efficacy is usually indication and disease specific and can include any methods known or available in the art for determining that a treatment is providing a beneficial effect to a patient. For example, evidence of treatment efficacy can include but is not limited to remission of the disease or indication. Further, treatment efficacy can also include general improvements in the overall health of the subject, such as but not limited to enhancement of patient life quality, increase in predicted subject survival rate, decrease in depression or decrease in rate of recurrence of the indication (increase in remission time). (See, e.g., Physicians' Desk Reference (2010).)

Glaucomas are a group of eye disorders characterized by progressive optic nerve damage in which an important part is a relative increase in intraocular pressure (IOP) that can lead to irreversible loss of vision. Glaucomas are categorized as open-angle glaucoma or angle-closure glaucoma. The “angle” refers to the angle formed by the junction of the iris and cornea at the periphery of the anterior chamber. The angle is where >98% of the aqueous humor exits the eye via either the trabecular meshwork and the Schlemm canal or the ciliary body face and choroidal vasculature. Glaucomas are further subdivided into primary (cause of outflow resistance or angle closure is unknown) and secondary (outflow resistance results from a known disorder), accounting for >20 adult types. Another group of glaucoma patients does not have IOP elevation, which in general is called normal tension glaucoma (NTG). NTG is also associated with progressive optic nerve degeneration and RGC death. Thus they are also subject to the therapeutic treatment of this disclosure.

Axons of retinal ganglion cells travel through the optic nerve carrying visual information from the eye to the brain. Damage to these axons causes ganglion cell death with resultant optic nerve atrophy and patchy vision loss. Elevated intraocular pressure (IOP; in unaffected eyes, the average range is 11 to 21 mm Hg) plays a role in axonal damage, either by direct nerve compression or diminution of blood flow. However, the relationship between externally measured pressure and nerve damage is complicated. Of people with IOP>21 mm Hg (ie, ocular hypertension), only about 1 to 2%/year (about 10% over 5 years) develop glaucoma. Additionally, about one third of patients with glaucoma do not have IOP>21 mm Hg (known as low-tension glaucoma or normal-tension glaucoma).

IOP is determined by the balance of aqueous secretion and drainage. Elevated IOP is caused by inhibited or obstructed outflow, not oversecretion; a combination of factors in the trabecular meshwork (eg, dysregulation of extracellular matrix, cytoskeletal abnormalities) appear to be involved. In open-angle glaucoma, IOP is elevated because outflow is inadequate despite an angle that appears unobstructed. In angle-closure glaucoma, IOP is elevated when a physical distortion of the peripheral iris mechanically blocks outflow.

Symptoms and signs of glaucoma vary with the type of glaucoma, but the defining characteristic is optic nerve damage as evidenced by an abnormal optic disk and certain types of visual field deficits. Glaucoma is diagnosed when characteristic findings of optic nerve damage are present and other causes have been excluded. Elevated IOP makes the diagnosis more likely, but elevated IOP can occur in the absence of glaucoma and is not essential for making the diagnosis.

The term “hydrogel” is used in its conventional sense to refer to a material that absorbs a solvent (e.g. water), undergoes swelling without measurable dissolution, and maintains three-dimensional networks capable of reversible deformation. “Swelling” as referred to herein is meant the isotropic expansion of the hydrogel structure as water molecules diffuse throughout the internal volume of the hydrogel. The properties of copolymer hydrogels disclosed herein may be modulated as desired, by varying the amounts of each component, ratios of each component or the density of specific components.

A suitable hydrogel composition may comprise a combination of biocompatible and water-absorbent polymers. These polymers can be natural, synthetic, or a combination thereof, depending on the desired properties and applications of the hydrogel. Examples of natural polymers include alginate, chitosan, hyaluronic acid, gelatin, and collagen, while synthetic polymers may include polyethylene glycol (PEG), polyacrylamide (PAAm), polyvinyl alcohol (PVA), or poly(N-isopropylacrylamide) (PNIPAM), among others.

The hydrogel composition can be tailored to exhibit specific characteristics such as mechanical strength, porosity, degradation rate, and responsiveness to external stimuli, including temperature, pH, or electric fields. These tunable properties enable the hydrogel to be customized for a wide range of applications.

Open cel polypropylene foam is a unique composition that consists of expanded polypropylene (EPP) with a distinctive open cell structure. The open cell foam is produced by expanding and foaming polypropylene beads or pellets, followed by molding or shaping to achieve the desired form. The resulting foam material exhibits a three-dimensional network of interconnected cells, creating a porous structure with open and interconnected voids throughout its volume. See, for example, WO1994013460A1 for information of making suitable foams, the disclosure of which is herein specifically incorporated by reference. The pore size of the foam may be from about 25 to 50 mM. Suitable foams ban be made, for example and without limitation, by mixing a suitable water soluble material suvh as sugar, salt crystals, etc. into polypropylene, followed by curing and dissolution.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) after the administration of a second therapeutic agent.

### Methods and Implants

As summarized above, aspects of the instant disclosure include methods of treating a subject for glaucoma, e.g. open-angle glaucoma or angle-closure glaucoma. The method comprises insertion of an implant suprachoroidal spacer, e.g. a monolithic implant, into the suprachoroidal space with a specially designed injector, parallel or perpendicular to the limbus. The suprachoroidal space expansion lowers IOP, and can remain in place for an extended period of time, e.g. for a period at at least about 1 year, 2 years, 3 years or more.

In some embodiments an individual selected for treatment has an IOP>21 mm Hg, termed ocular hypertension. In other embodiments the individual has an IOP that is elevated, but less than 21 mm Hg. The treatment of the disclosure may lower IOP by about 1 mm Hg, 2 mm, 3 mm, 4 mm, 5 mm, 7.5 mm, 10 mm, 12.5 mm, 15 mm or more. For example, an effective treatment may reduce other symptoms of glaucoma by about 10% or more, by about 20% or more, e.g., by 30% or more, by 40% or more, or by 50% or more, in some instances by 60% or more, by 70% or more, by 80% or more, or by 90% or more, for example, and may halt progression of the glaucoma in the subject. In some instances, an effective treatment will not only slow or halt the progression of the disease condition but will also induce the reversal of the condition.

In some embodiments the implant has final dimensions of from about 0.5 mm to about 1.5 mm diameter when hydrated, e.g. from about 0.5, 0.6, 0.7, 0.8, 0.9, 1 mm to about 1.5, 1.4, 1.3, 1.2, 1.1 mm. The length may be from about 5 mm to about 15 mm in length, e.g. from about 5, 6, 7, 8, 9, 10 mm, and up to about 15, 14, 13, 12, 11 mm. The shape my be cylindrical.

In some embodiments the material of an implant is an inert, biocompatible, water permeable material of appropriate rigidity. Suitable materials include, for example, hydrogels such as PEG methacrylate; polyvinyl alcohol (PVA); cellulose; collagen; polypropylene; polyurethane; PEEK; nylon; etc. In some embodiments the implant is a PVA hydrogel. In some embodiments the implant is an open cell polypropylene foam. In some embodiments the spacer implant is dip coated in sucrose, hyaluronic acid, polyvinyl alcohol, carboxmymethylcellulose, etc. to strengthen and/or facilitate insertion.

In some embodiments the implant is a metallic implant, e.g. formed of nitinol, steel, aluminum, and other biocompatible alloys. In some embodiments the implant is a plastic. In some such embodiments the implant may be coated, e.g. with an inert, biocompatible, water permeable material. In some embodiments the material of an implant is an inert, biocompatible, water permeable material of appropriate rigidity. Suitable materials include, for example, hydrogels such as PEG methacrylate, hyaluronic acid, etc; polyvinyl alcohol (PVA); cellulose; collagen; polypropylene; polyurethane; nylon; PLGA, PLA, polycaprolactone, etc. In some embodiments the implant is a PVA hydrogel. In some embodiments the implant is an open cell polypropylene foam. In some embodiments the spacer implant is dip coated in sucrose, hyaluronic acid, polyvinyl alcohol, carboxmymethylcellulose, etc. to strengthen and/or facilitate insertion.

In some embodiments an implant or an implant coating comprises a drug, e.g. for sustained release. Drugs of interest for this purpose may include, without limitation, β-blockers such as timolol, and betaxolol; alpha-agonists, such as brimonidine; carbonic anhydrase inhibitors such as dorzolamide, and brinzolamide; prostaglandin analogues such as latanoprost, bimatoprost, travoprost, tafluprost, latanoprostene bunod, etc.; rho kinase inhibitors, such as netarsudil; antibiotics such as moxifloxacin, cefalexin, cefazolin, etc.; enzymes such as protease, matrix metalloproteinase, collagenase, etc.; and the like.

The injector is designed to safely and reliably access the suprachoroidal space using a microneedle with length matched to the thickness of the sclera, for example a 27-gauge needle (OD 0.406 mm), which does not require suturing of the sclerotomy to prevent leakage and/or infection. The microneedle may be micromachined to a length appropriate for the insertion, e.g. from about 0.5 to about 1.5 mm in length. The baseplate may be angled, e.g. from about 45° to about 90°, usually about 65°. No cyclodialysis cleft is needed for this device to have its effect.

In some embodiments an injector device is provided, optionally including a pre-loaded spacer implant. In other embodiments, a kit comprising each of the injector device and spacer implants are provided, which kit may further comprise reagents and instructions for use.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. It is also understood that the terminology used herein is for the purposes of describing particular embodiments

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the appended claims.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Design and In Vitro Development of a Suprachoroidal Spacer Stent to Treat Glaucoma

A hydrogel implant that will not clear or degrade was designed to achieve long term IOP reduction, providing a suprachoroidal spacer stent to lower IOP.

### Materials and Methods

Design of suprachoroidal spacer implant. The suprachoroidal spacer implant is designed to be a monolithic implant that can delivered into the suprachoroidal space with a specially designed injector. The implant is water permeable so as to facilitate the free flow of water within the suprachoroidal space. The implant is rigid enough so as to easily advance within the suprachoroidal space but not so rigid to inadvertently penetrate through the choroid and retina. The injector is designed to safely and reliably access the suprachoroidal space using a microneedle with length matched to the thickness of the sclera. A 27-gauge needle (OD 0.406 mm) was chosen as the needle of choice as this is the largest commonly used needle size for intravitreal injections in the outpatient ophthalmology clinic. Such a needle size in commonly used and does not require suturing of the sclerotomy to prevent leakage and/or infection.

All materials are purchased from Sigma Aldrich until otherwise specified.

PEG hydrogel. Polyethylene glycol (PEG) was purchased from creative PEG works. Thirty percent (30% w/v) 10 kDa PEG with 4-arm methacrylate functional groups was solubilized in water. Lithium phenyl (2,4,6-trimethylbenzoyl) phosphinate (LAP) photo-initiator was added to the solution. The solution was then injection molded into 16 mm diameter blocks or silicone tubing with inner diameter of 0.5 mm. The molds were treated with ultraviolet light for 5 min to crosslink the PEG. A stainless-steel rod was used to push the PEG implant out of the mold. In some cases, the tubing was connected to the injector directly and the rod was used to deliver the implant through the injector.

Parameters were varied including arrangement of methacrylate functional groups (4-arm, 2-arm, 1-arm), molecular weight of PEG (5 kDa, 10 kDa, 20 kDa), weight percentage of PEG (10%, 20%, 30%, 40%), material of tubing (silicone tubing, PTFE tubing, steel tubing), inner diameter of tubing (0.3 mm, 0.5 mm, 1.0 mm).

PVA cryogel. Polyvinyl alcohol (PVA) was purchased from Sigma Aldrich. Twenty percent (20% w/v) PVA was dissolved in a 60% water/40% dimethyl sulfoxide (DMSO) solution that was heated to 90° C. PVA was injection molded into 16 mm blocks or stainless-steel tubing with inner diameter of 0.5 mm. The molds were subjected to 5 freeze-thaw cycles with 20 hours at −4° C. and 4 hrs at room temperature (nominally 20° C.). The molds were dried and demolded by pushing a stainless-steel rod through the tubing.

Parameters were varied including weight percentage of PVA (5%, 10%, 20%), DMSO concentration (0%, 10%, 20%, 40%, 100%), material of tubing (silicone tubing, PTFE tubing, steel tubing), number of freeze-thaw-cycles (0, 1, 3, 5).

Design of injector system. A 2-inch segment of 27-gauge 304 steel tubing (ID 0.305 mm, OD 0.406 mm; MicroGroup, Medway, MA) was used as the base of the injector. A 25° bias bevel was cut by wire electrical discharge machine (EDM) and polished on 400 grit sandpaper. One millimeter posterior to the needle tip, a stainless-steel washer (#000, ID 1.02 mm; McMaster-Carr, Santa Fe Springs, CA) was soldered to the tubing with stainless steel flux. Care was taken to protect the needle tip. A stainless-steel rod (0.279 mm diameter; MicroGroup, Medway, MA) was used as a plunger to deliver the implant through the bore of the needle. Parameters were varied including angle of bevel (25°, 45°), stainless steel washer diameter (#00, #000).

Mechanical testing of implant. Blocks of hydrogel bulk material were tested in compression on an Instron force displacement system using a protocol similar to the ATSM D695 standards (Compression of rigid plastics). Briefly, the PEG hydrogel and PVA cryogel were fabricated into a cylinder with diameter of 16 mm and height of 1-2 mm. The Instron was set to compress the material at a rate of 50 mm/min. Triplicate blocks of materials were tested in a hydrated state.

Because of the size of the implant, it was not possible to test the mechanical properties of the implant directly. Thus, the bend strength of the implant was determined. The implant was held 5 mm, 10 mm, 20 mm, and 30 mm away from the tip and applied perpendicularly to a balance scale. The maximum force was recorded for 5 segments. The same procedure was performed with the PEG hydrogel and PVA cryogel in their dehydrated and hydrated forms.

Parameters affecting implant insertion. To determine the ideal angle of insertion and length of needle, the following experiments were performed. Enucleated fresh rabbit eyes were obtained (Pel-Freez, Rogers, AR). The injector was applied to sclera until a pop was felt. A metal rod was introduced through the bore of the injector and slowly advanced. If strong resistance was felt and the injector came off the eye, the tip of the injector was had not yet entered the eye. If the rod could be easily advanced and a slight pop was felt (rod going through choroid/retina), the tip of the injector was considered to be in the suprachoroidal space. If the rod could be easily advanced and no pop was felt and the rod was visible within the eye, the tip of the injector was considered to be in the vitreous. Six replicates were performed for each condition.

Injector force of insertion. To determine the force needed to penetrate through the choroid/retina, the following was performed to indirectly determine the force needed by proxy. Prolene sutures of different sizes (2-0, 4-0, 5-0, 6-0, 7-0, 8-0, 9-0, 10-0; Ethicon, Bridgewater, NJ) was held 5 mm, 10 mm, 20 mm, and 30 mm away from the tip and applied perpendicularly to a balance scale. The maximum force was recorded for 5 segments. Fresh enucleated rabbit eyes were prepared, and a 27 G microneedle with length of 0.6 mm was applied to the sclera perpendicularly until a pop was felt. Different prolene sutures were introduced through the bore of the microneedle. If a slight pop was felt (suture going through choroid/retina), this prolene suture was recorded as being able to penetrate through the choroid/retina. If no pop was felt with the prolene suture, this suture was recorded as being unable to penetrate through the choroid/retina. If the suture could not be advanced, another sclerotomy was made and this was not considered a replicate.

Ex vivo delivery of implant. Prior to deploying the implant, the PVA cryogel was made as previously described and loaded into the injector as previously described. To aid in identification of the implant, 1 mg/ml fluorescein was added to the PVA cryogel solution. Enucleated fresh rabbit eyes were obtained (Pel-Freez, Rogers, AR). The injector was applied to sclera until a pop was felt. A metal rod was depressed to advance the implant into the suprachoroidal space. The injector was removed. In some cases, ultrasound biomicroscopy was performed to visualize the implant in the suprachoroidal space. The eye was then flash frozen in ethanol chilled over dry ice. The eye was cut open in a petal fashion as previously described. The vitreous was isolated. Fluorescent images were acquired to determine the location of the implant.

### Results and Discussions

Mechanical testing of PEG hydrogel. We initially chose to use a PEG hydrogel due to its known biocompatibility, ease of processing, and transparency. We fabricated PEG hydrogels with different parameters and tested their mechanical properties in compression (FIG. 2). In general, the PEG blocks broke into small pieces upon compression (FIG. 2B).

Because of the small size of the spacer implant, direct mechanical testing was not possible. Thus, bend strength of PEG implants with different parameters was determined (Table 1). The parameters that seemed to impart the greatest rigidity to the PEG implants was a diameter of 1.0 mm and dehydrating the implant. Upon contact with water, the PEG implants rapidly hydrated within seconds and lost the mechanical rigidity of the dehydrated implants. No PEG implants with diameter of 0.3 mm were able register a force before bending. Thus, it was unlikely that PEG hydrogel implants would be able to be advanced within the suprachoroidal space since the material was not rigid enough.

TABLE 1
Bending force of select PEG hydrogels with length of 30
mm, 20 mm, 10 mm, and 5 mm. Mean ± SD for triplicates.

| | Hydrogel | | | Force at | Force at | Force at | Force at |
|---|---|---|---|---|---|---|---|
| Diameter | conc | Dipcoating | State | 30 mm (mg) | 20 mm (mg) | 10 mm (mg) | 5 mm (mg) |
| 1.0 mm | 50% | Viscoat | Wet | 0 ± 0 | 210 ± 13 | 722 ± 153 | 1928 ± 567 |
| 1.0 mm | 50% | Viscoat | Dry | 0 ± 0 | 5753 ± 2480 | 11066 ± 3447 | 16590 ± 1212 |
| 1.0 mm | 50% | None | Wet | 267 ± 30 | 331 ± 63 | 908 ± 197 | 3697 ± 522 |
| 1.0 mm | 50% | None | Dry | 0 ± 0 | 1727 ± 436 | 3497 ± 393 | 6091 ± 2703 |
| 0.5 mm | 30% | 2% CMC | Wet | 0 ± 0 | 0 ± 0 | 19 ± 1 | 61 ± 15 |
| 0.5 mm | 30% | 2% CMC | Dry | 0 ± 0 | 0 ± 0 | 33 ± 6 | 43 ± 2 |
| 0.5 mm | 50% | None | Wet | 11 ± 6 | 11 ± 5 | 33 ± 11 | 55 ± 11 |
| 0.5 mm | 50% | None | Dry | 0 ± 0 | 26 ± 2 | 44 ± 4 | 71 ± 4 |
| 0.3 mm | 40% | Viscoat | Wet | 0 ± 0 | 0 ± 0 | 0 ± 0 | 0 ± 0 |
| 0.3 mm | 40% | Viscoat | Dry | 0 ± 0 | 0 ± 0 | 0 ± 0 | 0 ± 0 |
| 0.3 mm | 40% | None | Wet | 0 ± 0 | 0 ± 0 | 0 ± 0 | 0 ± 0 |
| 0.3 mm | 40% | None | Dry | 0 ± 0 | 0 ± 0 | 0 ± 0 | 0 ± 0 |

Mechanical testing of PVA cryogel. PVA cryogels share many of the same characteristics as the PEG hydrogel, including biocompatibility, ease of processing, and transparency. We determined the mechanical properties of PVA cryogels in compression. In general, the PVA blocks fractured upon compression. The PVA cryogels had much greater rigidity than the PEG hydrogel.

Because of the small size of the spacer implant, direct mechanical testing was not possible. Thus, bend strength of PVA implants with different parameters was determined. Because the most rigid configuration of PVA cryogel was significantly greater than the force needed to penetrate the choroid/retina, the parameters of the PVA cryogel were tuned to enable easy advancement within the suprachoroidal space but not penetrate the through the choroid/retina.

Injector angle of insertion. To determine the ideal length and insertion angle of the needle, we performed insertion studies in ex vivo rabbit eyes. A needle length of 1.0 mm at an oblique angle was able to reliable enter the suprachoroidal space. Such an oblique angle would enable delivery of the spacer implant without kinking.

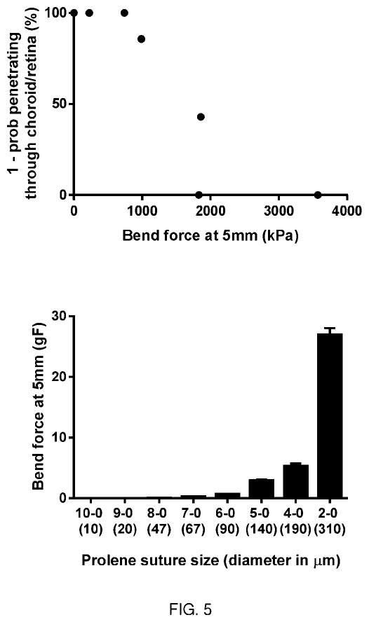

Force to penetrate choroid and retina. To improve the safety of the spacer implant, the force required to penetrate the choroid/retina was determined (FIG. 5A). First, the probability of different sized prolene sutures penetrating through the choroid/retina was found. Prolene sutures designated 6-0 (diameter 90 μm) were unlikely to penetrate choroid/retina, and 7-0 (diameter 67 μm) never penetrated in our study. Then the bend force of these sutures was also determined. (FIG. 5B). The 7-0 prolene suture had a bend force of 0.262±0.035 gF when held 5 mm from the tip. This force was used to choose the ideal PVA cryogel parameters.

Insertion of PVA cryogel implants into suprachoroidal space. The ability of the custom designed injector to deliver the spacer implant into the suprachoroidal space was determined. A subset of PVA cryogel implants was tested and as predicted there was a high probability of success. A subset of PEG hydrogel implants was tested and as predicted with mechanical testing, none of the implants could be delivered into the suprachoroidal space.

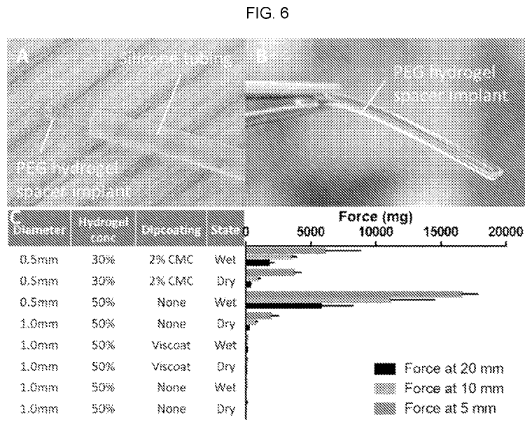

Prototype hydrogel spacer implants, such as a PEG hydrogel implant described here, were tested. Briefly, PEG-methacrylate and lithium phenyl-2, 4, 6-trimethylbenzoylphospinate (LAP) photo-initiator was dissolved in PBS and injected into silicone tubing. A UV light source was used to cure the PEG-methacrylate for 5 min, and the hydrogel was extruded from the silicone tubing, washed, and dried (FIGS. 6A and B). Some parameters have already been optimized, including PEG-methacrylate chemical structure (linear, 4-arm, 8-arm), molecular weight of PEG (3, 5, 10, 20 kDa), PEG w/v % concentration (10, 20, 30, 40, 50, 75%), and silicone tubing inner diameter (0.3, 0.5, 1 mm). To further strengthen the implant and facilitate insertion, the spacer implant was dip coated in sucrose, hyaluronic acid, polyvinyl alcohol, or carboxmymethylcellulose. Mechanical testing (imagine Cochet-Bonnet esthesiometry) has also been performed (FIG. 6C).

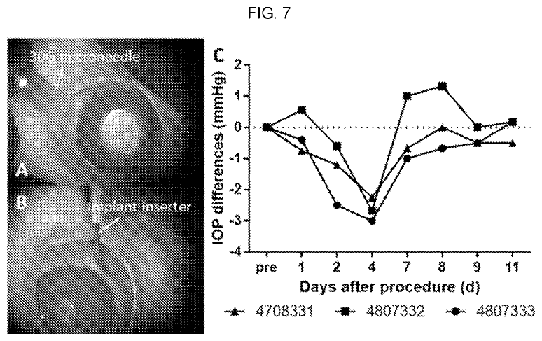

Spacer implants were successfully implanted in live rabbits. After inducing general anesthesia, tetracaine was applied. A 30 G microneedle was used to access the SCS and inject ophthalmic viscoelastic device. Hydrogel and polypropylene spacer implants were then delivered into the SCS with a microneedle inserter (FIGS. 7A and B). Animals were monitored every 1-3 days and IOP was measured by rebound tonometry. IOP reduction was seen in the treated eyes.

Solid polypropylene implants are expected to be impermeable to water, whereas open cell polypropylene structures are highly permeable to water. Hydrogels are, by definition, comprised of water, but are actually not as permeable as open cell polypropylene due to the strong hydrogen bonding between the water and hydrogel. Our approach is to insert spacer implants made of PEG hydrogel, solid polypropylene, or open cell polypropylene foam into the SCS, and serially measure IOP after implantation. Terminal anesthesia is induced after 14 days, and fluorescein is injected into the anterior chamber so that clearance kinetics and routes of fluorescein clearance can be determined. Eye fluids will be carefully collected from the scleral surface and from a transected vortex vein over 1 hr so that clearance kinetics and relative contributions of different routes of clearance from the SCS can be calculated. The primary endpoint is IOP reduction versus untreated fellow eye at 14 days for hydrogel, solid polypropylene, and open-cell polypropylene foam implants. Secondary endpoint is an understanding of clearance kinetics and routes of clearance of aqueous humor after SCS expansion.

The anterior border of the SCS is in contact with the ciliary body, which is responsible for aqueous humor production. Ciliary body shutdown may be partially responsible for IOP reduction with SCS expansion. Placement adjacent to the ciliary body (i.e., parallel to the limbus) may result in maximal IOP reduction by a combination of reduced aqueous production and improved uveoscleral outflow. Spacer implants are inserted parallel or perpendicular to the limbus, and serially measure IOP after implantation for 28 days. Implant positioning within the SCS is tracked weekly with fundus photography and ultrasound biomicroscopy. The primary endpoint is IOP reduction versus untreated fellow eye at 28 days for spacer implants placed parallel vs perpendicular to the limbus. Secondary endpoints include effect of implant diameter and implant morphology on IOP reduction.

There is limited knowledge on the biocompatibility of materials within SCS. Our approach is to insert spacer implants made of PEG hydrogel, cellulose, collagen, polypropylene, polyurethane, PEEK, or nylon into the SCS of rabbits, and serially measure IOP for 90 days after implantation. Animals are euthanized at 1 wk and at 90 d to look for short- and long-term responses. For each treated eye, H&E staining is done to quantify the cross-sectional surface area of the scar extending from the implant and number of immune cells within 1 mm of the implant. The primary endpoint is IOP reduction versus untreated fellow eye at 3 months and degree of scar formation around each implant material.

Glaucoma is a leading cause of blindness, and IOP reduction can slow glaucoma progression. In this study, we describe the development of a monolithic hydrogel suprachoroidal spacer implant to treat glaucoma. PVA cryogel was chosen as the material for the implant due to its mechanical properties, biocompatibility, and transparency. To determine the ideal cryogel parameters, we needed to balance the rigidity of the implant such that it could be easily advanced within the suprachoroidal space but not penetrate through the choroid/retina. The suprachoroidal space is a potential space between the sclera and the choroid held together by fibrils, and thus a nominal force is required to part the tissue. For the upper limit, we used prolene suture as a proxy to infer the mechanical rigidity at which the choroid/retina could not be penetrated, which corresponded to a bend force of 0.262±0.035 gF when held 5 mm from the tip. PVA cryogels with a bend force less than that was chosen. As predicted, PVA cryogel that fulfilled these criteria could be deployed within the suprachoroidal space.

We also developed a custom-designed injector system to safely deliver the implant into the suprachoroidal space. The diameter of the needle was chosen as 27-gauge since this is the largest needle size used for intravitreal injections in the outpatient ophthalmology clinic. No additional sutures are needed to close the tissue after an injection with a needle of this size. Microneedle-based drug delivery systems to access the suprachoroidal space are FDA approved for use in ophthalmology. The length of the needle is chosen to match the thickness of the sclera. Microneedles used in suprachoroidal drug delivery are held perpendicular to the sclera and the injected fluid formulation can navigate this angle. Because the implant should be delivered without kinking, an oblique needle angle and thus overall needle design needed to be investigated. To our knowledge, this is the first use of a microneedle to deliver a medical device into the suprachoroidal space.

Chiang et al and Chae et al also showed that suprachoroidal expansion with a fluid formulation can lower IOP. However, it is difficult to control the extent and direction of suprachoroidal expansion with a liquid formulation. A solid monolithic implant ia easier to implant with fewer regulatory hurdles than a liquid formulation.

In summary, we have developed a suprachoroidal space implant and injector to lower IOP and treat glaucoma.

### WORKS CITED

Tham Y C, Li X, Wong T Y, Quigley H A, Aung T, Cheng C Y. Global prevalence of glaucoma and projections of glaucoma burden through 2040: a systematic review and meta-analysis. Ophthalmology. November 2014; 121(11):2081-90. doi:10.1016/j.ophtha.2014.05.013
Goel M, Picciani R G, Lee R K, Bhattacharya S K. Aqueous humor dynamics: a review. Open Ophthalmol J. September 2010; 4:52-9. doi:10.2174/1874364101004010052
Kass M A, Heuer D K, Higginbotham E J, et al. The Ocular Hypertension Treatment Study: a randomized trial determines that topical ocular hypotensive medication delays or prevents the onset of primary open-angle glaucoma. Arch Ophthalmol. June 2002; 120(6):701-13; discussion 829-30. doi:10.1001/archopht.120.6.701
Leske M C, Heijl A, Hyman L, et al. Predictors of long-term progression in the early manifest glaucoma trial. Ophthalmology. November 2007; 114(11):1965-72. doi:10.1016/j.ophtha.2007.03.016
Heijl A, Leske M C, Bengtsson B, Hyman L, Hussein M, Group EMGT. Reduction of intraocular pressure and glaucoma progression: results from the Early Manifest Glaucoma Trial. Arch Ophthalmol. October 2002; 120(10):1268-79. doi:10.1001/archopht.120.10.1268
Camras C B. Comparison of latanoprost and timolol in patients with ocular hypertension and glaucoma: a six-month masked, multicenter trial in the United States. The United States Latanoprost Study Group. Ophthalmology. January 1996; 103(1):138-47. doi:10.1016/s0161-6420(96)30749-5
Watson P, Stjernschantz J. A six-month, randomized, double-masked study comparing latanoprost with timolol in open-angle glaucoma and ocular hypertension. The Latanoprost Study Group. Ophthalmology. January 1996; 103(1):126-37. doi:10.1016/s0161-6420(96)30750-1
Zimmerman T J, Kaufman H E. Timolol. A beta-adrenergic blocking agent for the treatment of glaucoma. Arch Ophthalmol. April 1977; 95(4):601-4. doi:10.1001/archopht.1977.04450040067008
Ghate D, Edelhauser H F. Barriers to glaucoma drug delivery. J Glaucoma. March 2008; 17(2):147-56. doi:10.1097/IJG.0b013e31814b990d
Taylor S A, Gabraith S M, Mills R P. Causes of non-compliance with drug regimens in glaucoma patients: a qualitative study. J Ocul Pharmacol Ther. October 2002; 18(5):401-9. doi:10.1089/10807680260362687
Gazzard G, Konstantakopoulou E, Garway-Heath D, et al. Selective laser trabeculoplasty versus eye drops for first-line treatment of ocular hypertension and glaucoma (LiGHT): a multicentre randomised controlled trial. Lancet. April 2019; 393(10180):1505-1516. doi:10.1016/S0140-6736(18)32213-X
Chiang B, Kim Y C, Doty A C, Grossniklaus H E, Schwendeman S P, Prausnitz M R. Sustained reduction of intraocular pressure by supracilary delivery of brimonidine-loaded poly(lactic acid) microspheres for the treatment of glaucoma. J Control Release. April 2016:228:48-57. doi:10.1016/j.jconrel.2016.02.041
Chae J J, Jung J H, Zhu W, et al. Drug-Free, Nonsurgical Reduction of Intraocular Pressure for Four Months after Suprachoroidal Injection of Hyaluronic Acid Hydrogel. Advanced Science. Jan. 1, 2021 2021; 8(2):2001908. doi:10.1002/advs.202001908
Pederson J E, Gaasterland D E, MacLellan H M. Experimental ciliochoroidal detachment. Effect on intraocular pressure and aqueous humor flow. Arch Ophthalmol. March 1979; 97(3):536-41. doi:10.1001/archopht.1979.01020010280020
Chiang B, Jung J H, Prausnitz M R. The suprachoroidal space as a route of administration to the posterior segment of the eye. Adv Drug Deliv Rev. February 2018; 126:58-66. doi:10.1016/j.addr.2018.03.001
Chiang B, Kim Y C, Edelhauser H F, Prausnitz M R. Circumferential flow of particles in the suprachoroidal space is impeded by the posterior ciliary arteries. Exp Eye Res. 04 2016; 145:424-431. doi:10.1016/j.exer.2016.03.008
Chiang B, Venugopal N, Grossniklaus H E, Jung J H, Edelhauser H F, Prausnitz M R. Thickness and Closure Kinetics of the Suprachoroidal Space Following Microneedle Injection of Liquid Formulations. Invest Ophthalmol Vis Sci. January 2017; 58(1):555-564. doi:10.1167/iovs.16-20377
Clearside Biomedical. XIPERE™ (triamcinolone acetonide injectable suspension), for suprachoroidal use [package insert]. U.S. Food and Drug Administration website. Revised: 10/2021. Accessed: August/2022.
Patel S R, Berezovsky D E, McCarey B E, Zarnitsyn V, Edelhauser H F, Prausnitz M R. Targeted administration into the suprachoroidal space using a microneedle for drug delivery to the posterior segment of the eye. Invest Ophthalmol Vis Sci. July 2012; 53(8):4433-41. doi:10.1167/iovs.12-9872
Patel S R, Lin A S, Edelhauser H F, Prausnitz M R. Suprachoroidal drug delivery to the back of the eye using hollow microneedles. Pharm Res. January 2011; 28(1):166-76. doi:10.1007/si1095-010-0271-y

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method of lowering intraocular pressure (IOP) in an individual with ocular hypertension or glaucoma, the method comprising:
inserting a suprachoroidal spacer implant into the suprachoroidal space of the individual, wherein the spacer induces suprachoroidal space expansion and lowers IOP.

2. The method of claim 1, wherein the implant is rigid enough so as to easily advance within the suprachoroidal space but not so rigid to inadvertently penetrate through the choroid and retina.

3. The method of claim 1, wherein the implant is a water permeable material, optionally a hydrogel.

4. The method of claim 1, wherein the implant is an inert, biocompatible, water permeable material of appropriate rigidity.

5. (canceled)

6. The method of claim 1, wherein the implant is a material selected from PEG, PEG methacrylate; hyaluronic acid; polyvinyl alcohol (PVA); cellulose; collagen; polypropylene; polyurethane; PLGA, PLA, polycaprolactone; and nylon.

7. (canceled)

8. The method of claim 1, wherein the implant is an open cell polypropylene foam.

9. The method of claim 1, wherein the implant is metal.

10. The method of claim 1, wherein the implant comprises a biocompatible coating.

11. The method of claim 1, wherein the implant comprises a drug, optionally selected from one or more of a b-blocker, alpha-agonist, carbonic anhydrase inhibitor, prostaglandin analogue, rho kinase inhibitor, antibiotic and enzyme.

12. (canceled)

13. The method of claim 1, wherein the implant has final dimensions of from about 0.3 mm to about 1.5 mm diameter when hydrated and a length of from about 5 mm to about 15 mm.

14. The method of any of claims 1-13 wherein the implant is inserted 1-5 mm posterior to limbus, optionally wherein the implant is inserted 2-3 mm posterior to limbus.

15. (canceled)

16. The method of claim 1 wherein the implant is inserted parallel to the limbus or perpendicular to the limbus.

17. (canceled)

18. The method of claim 1, wherein the implant is inserted with an injector, optionally where wherein the injector comprises a needle of a size that does not require suturing of the sclerotomy, micromachined to a length appropriate for the insertion with an angled baseplate, optionally wherein the needle length of the injector is from 0.5 to 1.5 mm, optionally wherein the needle inner diameter is from 0.25 to 0.6 mm.

19-20. (canceled)

21. The method of claim 18, wherein the base plate of the injector is angled at 45-60 degrees.

22-23. (canceled)

24. The method of claim 18, wherein:
(a). the microneedle extends distally from the body of the injector at an angle of 45 to 60 degrees such that the microneedle inserts through the sclera at the angle:
(b) wherein the microneedle is mounted on a distal face of the device, and wherein the microneedle extends at an angle of 45 to 60 degrees from and relative to the distal face; or
(c) wherein the microneedle is mounted on a distal face of the device, wherein the microneedle extends substantially perpendicular from and relative to the distal face, and wherein the distal face comprises an angle of 45 to 60 degrees relative to the body of the device.

25-26. (canceled)

27. An injector for use in the method of claim 18.

28. A kit for use in the method of claim 1.
